(19) United States
(12) Patent Application Publication
(10) Pub. No.: US 2018/0249929 A1
(43) Pub. Date: Sep. 6, 2018

(54) BREATH SENSOR

(71) Applicants: NGK SPARK PLUG CO., LTD., Nagoya-shi (JP); SPIROSURE, INC., Pleasanton, CA (US)

(72) Inventors: Shinichi NAKAGAWA, Konan-shi (JP); Tatsunori ITO, Inazawa-shi (JP); Hiroyuki NISHIYAMA, Konan-shi (JP); Masahiro TAKAKURA, Komaki-shi (JP); Masatoshi UEKI, Niwa-gun (JP); Solomon SSENYANGE, Fremont, CA (US); Ryan LEARD, Oakland, CA (US)

(73) Assignees: NGK SPARK PLUG CO., LTD., Nagoya-shi (JP); SPIROSURE, INC., Pleasanton, CA (US)

(21) Appl. No.: 15/448,672

(22) Filed: Mar. 3, 2017

(51) Int. Cl. A61B 5/08; A61B 5/097; G01N 33/497

(52) U.S. Cl. CPC A61B 5/082; G01N 2033/4975; G01N 33/497; A61B 5/097

(57) ABSTRACT

A breath sensor including a main body into which breath can be introduced, a conversion section for converting a gas component contained in the breath to a specific component, and a sensing section for detecting the specific component. The sensing section constitutes a first unit including an integrated detection element and a first heater. The conversion section constitutes a second unit including an integrated catalyst and a second heater. The first unit and the second unit are disposed inside the main body so as to be separated from each other with a gap therebetween, or are in contact with each other via a heat insulating member. The first unit and second unit are in communication with each other through a pipe that allows the breath to pass therethrough, and the pipe is partially bent.

### BACKGROUND OF THE INVENTION

### Field of the Invention

The present invention relates to a breath sensor for detecting the concentration of a specific component contained in breath.

### Description of the Related Art

One known sensor for diagnosis of, for example, asthma measures NOx contained at a very low concentration (at a level of several ppb to several hundreds of ppb) in breath (see U.S. Patent Application Publication No. 2015/0250408 incorporated herein by reference in its entirety, including but not limited to, FIG. 6B).

In this sensor, a conversion section including a PtY (platinum-zeolite) catalyst for converting NO in breath to NO₂ and a sensing section including a mixed-potential sensor element for detecting NO₂ are formed as a single unit using a ceramic stacking technique.

A heater for heating the catalyst is disposed in the conversion section, and a heater for heating the sensor element is disposed in the sensing section. The temperatures of these heaters are controlled separately. This is because the temperature for optimal operation of the catalyst is different from the temperature for optimal operation of the sensor element.

When the conversion section and the sensing section are formed as a single unit, the electromotive force (element output) of the sensor element may decrease each time the measurement is repeated. This may be because heat generated by the heater for activation of the catalyst affects the control of the temperature of the sensor element disposed close to the catalyst so as to influence the element output.

### SUMMARY OF THE INVENTION

It is therefore an object of the present invention to provide a breath sensor in which the influence of the heat from the conversion section on the sensing section is reduced, to thereby improve the accuracy of detection of a specific component by the sensing section.

The above object has been achieved by providing, (1) a breath sensor which comprises a main body into which breath can be introduced, a conversion section that converts a gas component contained in the breath to a specific component, and a sensing section that detects the specific component contained in the breath passing through the conversion section, the conversion section and the sensing section being disposed inside the main body. The sensing section constitutes a first unit including an integrated detection element and a first heater, the detection element having electric characteristics that vary depending on the concentration of the specific component, and the first heater heating the detection element to a first temperature. The conversion section constitutes a second unit including an integrated catalyst and a second heater, the catalyst converting the gas component contained in the breath, and the second heater heating the catalyst to a second temperature different from the first temperature. The first unit and the second unit are disposed inside the main body so as to be separated from each other with a gap therebetween, or are in contact with each other via a heat insulating member. The first unit and second unit are in communication with each other through a pipe that allows the breath to pass therethrough, and the pipe is partially bent.

In the breath sensor (1), the first unit including the detection element and the first heater that are integrated together, and the second unit including the catalyst and the second heater that are integrated together, are provided as single units. The first unit and the second unit are disposed inside the main body so as to be separated from each other with a gap therebetween, or are in contact with each other through the heat insulating member. Heat is applied to the catalyst from the second heater in order to activate the catalyst. However, with the above configuration, the influence of the heat on the sensing section is reduced, so that the detection accuracy of the sensing section can be improved.

Since the pipe is partially bent, the path length of the pipe is longer than that of an unbent pipe. In this case, the breath heated in the conversion section is easily cooled within the pipe. This can also reduce the influence of the heat applied to the conversion section on the sensing section.

In a preferred embodiment (2) of the breath sensor (1) above, the pipe is disposed inside the main body without being exposed outside the main body.

The breath sensor (2) is less susceptible to external perturbations (such as wind). Therefore, the temperature of the cooled gas is stabilized, so that the output can be stabilized.

In the present invention, the first heater heats the detection element to the first temperature, and the second heater heats the catalyst to the second temperature different from the first temperature. Even in this case, the influence of the heat in the conversion section on the sensing section is reduced, and the breath sensor thus obtained has improved accuracy of detection of the specific component.

### BRIEF DESCRIPTION OF THE DRAWINGS

FIG. 1 is a perspective view of a breath sensor according to an embodiment of the present invention;

FIG. 2 is a cross-sectional view of the breath sensor taken along line A-A in FIG. 1;

FIG. 3 is an exploded perspective view of the breath sensor;

FIG. 4 is an exploded perspective view of a sensing section; and

FIG. 5 is an exploded perspective view of a conversion section.

### DESCRIPTION OF THE PREFERRED EMBODIMENTS

The present invention will next be described in detail with reference to the drawings. However, the present invention should not be construed as being limited thereto.

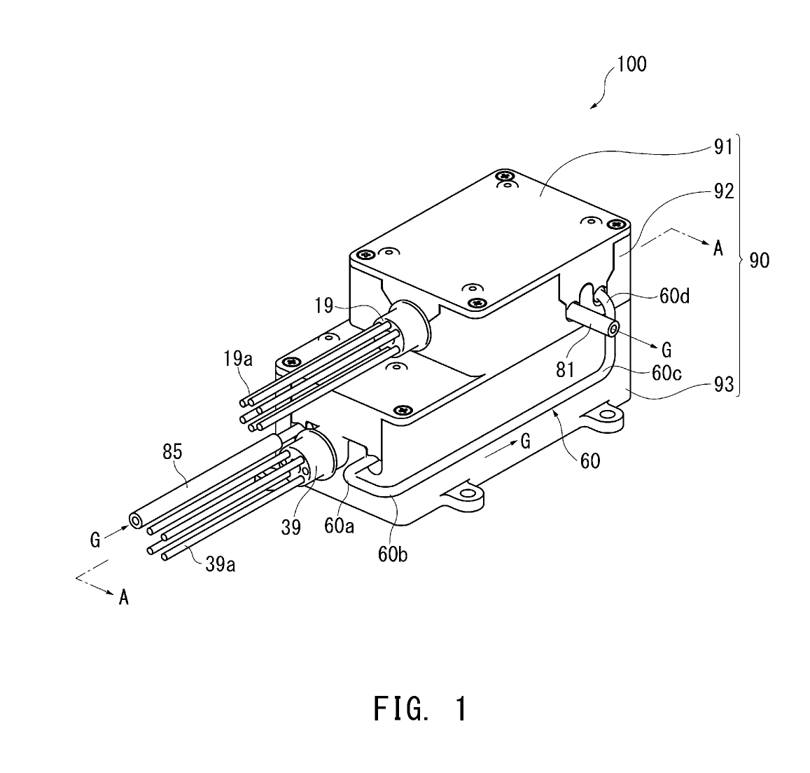

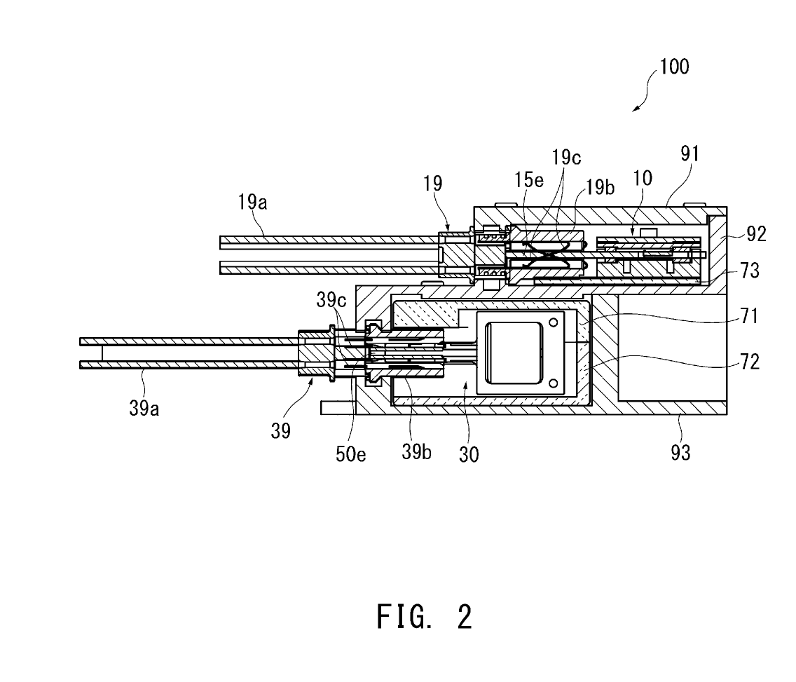

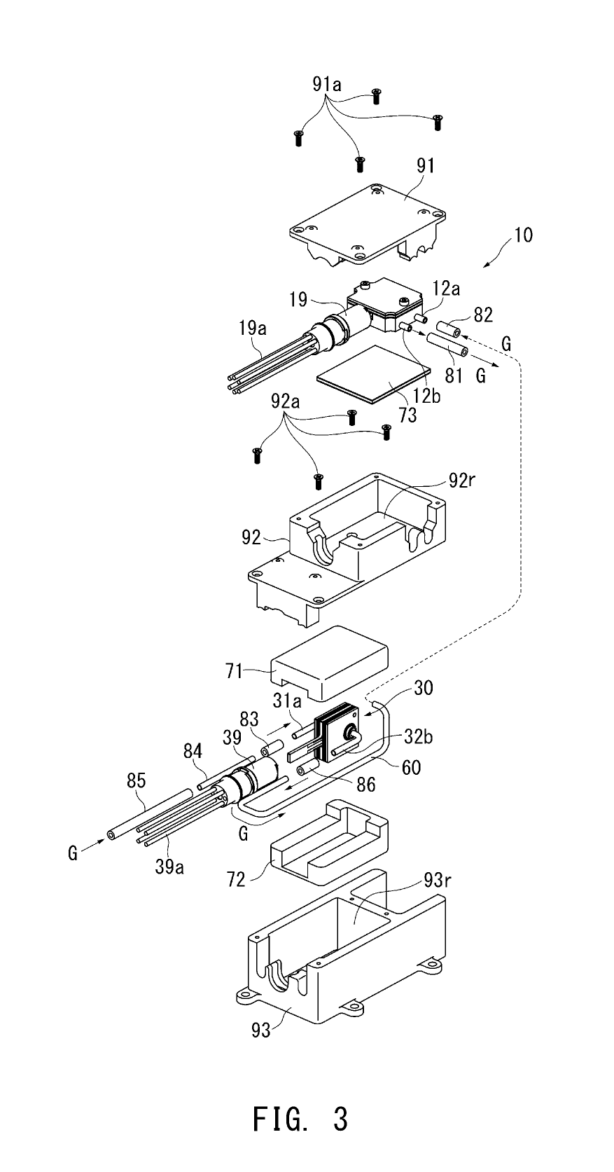

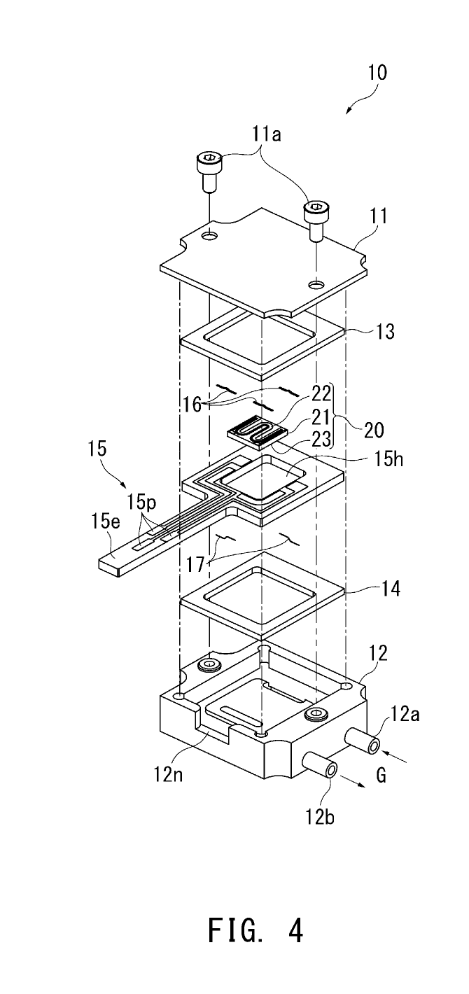

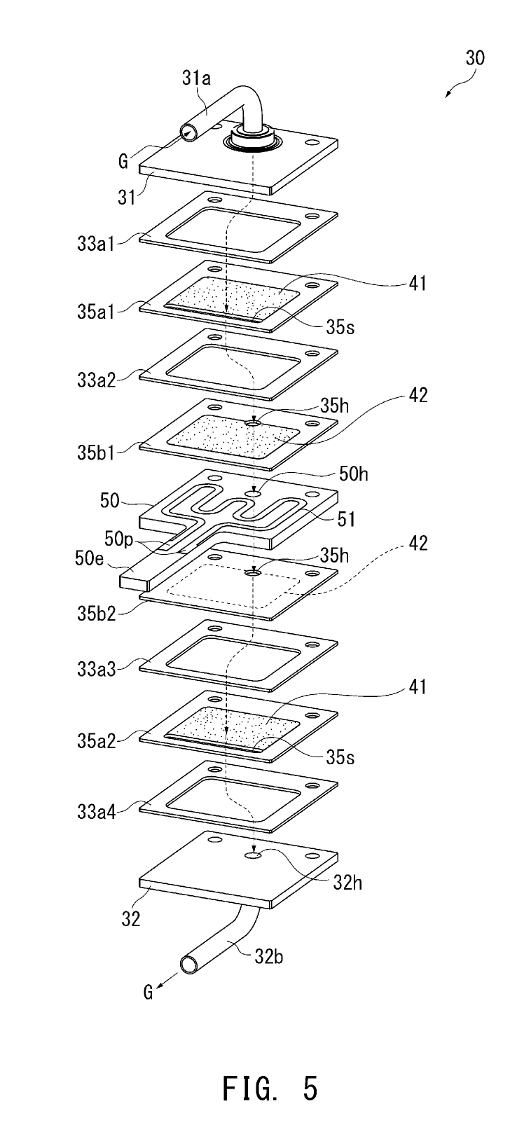

FIG. 1 is a perspective view of a breath sensor 100 according to an embodiment of the present invention, and FIG. 2 is a cross-sectional view of the breath sensor 100 taken along line A-A in FIG. 1. FIG. 3 is an exploded perspective view of the breath sensor 100, and FIG. 4 is an exploded perspective view of a sensing section 10. FIG. 5 is an exploded perspective view of a conversion section 30.

As shown in FIGS. 1 and 2, the breath sensor 100 includes a main body 90 serving as a housing, the sensing section 10, the conversion section 30, and a pipe (gas circulation pipe) 60. The sensing section 10 and the conversion section 30 are contained in the main body 90, and the main body 90 as a whole is formed into a box shape.

The main body 90 includes: a base 93 having an approximately rectangular shape and elongated in the left-right direction in FIG. 1; an upper case 92 having an approximately rectangular shape and shorter in the left-right direction in FIG. 1 than the base 93; and a lid 91 fastened to the upper case 92 with screws 91a to close an internal space 92r of the upper case 92 (see FIG. 3). The main body 90 is formed of a metal or a resin.

One longitudinal end of the upper case 92 (the right end in FIG. 1) is aligned with one longitudinal end of the base 93 (the right end in FIG. 1), and the upper case 92 is fastened to the upper surface of the base 93 with screws 92a to thereby close an internal space 93r of the base 93 (see FIG. 3).

As shown in FIG. 3, the sensing section 10 is contained in the internal space 92r of the upper case 92, and a tubular cassette connector 19 is connected to the sensing section 10. The conversion section 30 is contained in the internal space 93r of the base 93, and a tubular cassette connector 39 is connected to the conversion section 30.

A detection output for a specific component from the sensing section 10 is output to the outside from one end of the cassette connector 19 (the left end in FIG. 1) through lead wires 19a, and heater power for energizing a heater included in the sensing section 10 is externally supplied through lead wires 19a. Heater power for heating the conversion section 30 is externally supplied to one end of the cassette connector 39 (the left end in FIG. 1) through lead wires 39a.

As shown in FIG. 1, breath G is introduced into the conversion section 30 inside the base 93 through a sub-pipe 85, discharged from the conversion section 30, and then introduced into the sensing section 10 inside the upper case 92 by way of the main pipe 60 provided outside the base 93. The sensing section 10 detects a specific component in the breath G, and the breath G is discharged to the outside through a sub-pipe 81 provided outside the upper case 92.

The main pipe 60 protrudes from a front face of the base 93 (the left face in FIG. 1), is bent at a bent portion 60a 90° in the direction of the width of the base 93 (an oblique direction toward the lower right side in FIG. 1), further bent at a bent portion 60b 90° in the lengthwise direction of the base 93 (the direction toward the right side in FIG. 1), and then extends in the lengthwise direction of the base 93. Near the one longitudinal end of the base 93 (the right end in FIG. 1), the main pipe 60 is bent at a bent portion 60c 90° in an upward direction (the upward direction in FIG. 1) toward the upper case 92, bent at a bent portion 60d 90° in the direction of the width of the upper case 92 (an oblique direction toward the upper side in FIG. 1), and then enters the upper case 92.

As described above, the main pipe 60 has at least one bent portion (four bent portions in this example, i.e., the bent portions 60a to 60d). The main pipe 60 is formed from a metal-made pipe (e.g., a stainless steel alloy pipe) having high heat dissipation performance.

The main pipe 60 corresponds to the “pipe” of the invention.

Referring next to FIG. 4, the sensing section 10 will be described.

The sensing section 10 includes: a metal-made lower case 12 having an approximately rectangular box shape and having a recess on its upper surface (the surface facing upward in FIG. 4); a lid 11 for closing the recess of the lower case 12; a ceramic circuit board 15 contained in the lower case 12; rectangular frame-shaped seal members (gaskets) 13 and 14; an element unit 20 disposed within an opening 15h of the ceramic circuit board 15; and energizing members 16 and 17 for suspending and fixing the element unit 20 within the opening 15h. The ceramic circuit board 15 has a shape including a rectangular plate-shaped portion and a narrow width base end portion 15e protruding from one edge of the rectangular plate-shaped portion.

The seal members 13 and 14 and a forward end portion of the ceramic circuit board 15 are contained in the internal space of the lower case 12, and the narrow width base end portion 15e of the ceramic circuit board 15 protrudes outside the lower case 12 through a notch 12n of the lower case 12.

The lid 11 is disposed on the seal member 13 and fastened to the lower case 12 with bolts 11a. As a result, the seal members 13 and 14 are pressed between the lower case 12 and the lid 11, and the ceramic circuit board 15 is thereby sealed.

An introduction pipe 12a and a discharge pipe 12b for the breath G are attached to one side wall of the lower case 12 (the right side wall in FIG. 4). The breath G introduced into the lower case 12 through the introduction pipe 12a comes into contact with the element unit 20, and the concentration of a specific component is detected. Then the breath G is discharged to the outside through the discharge pipe 12b.

The element unit 20 has an approximately rectangular plate shape and includes a substrate 21, a first heater 22 disposed on an upper surface of the substrate 21 (the surface facing upward in FIG. 4), and a detection element 23 disposed on a lower surface of the substrate 21. The element unit 20 has an integral structure in which the detection element 23 and the first heater 22 are stacked on the lower and upper surfaces, respectively, of the substrate 21.

The electric characteristics of the detection element 23 vary depending on the concentration of the specific component, and the change in the electric signal is sensed to detect the concentration of the specific component. When the first heater 22 is energized, heat is generated, and the detection element 23 is thereby heated to a first temperature, which is the operating temperature of the detection element 23. Output terminals of the detection element 23 and energization terminals of the first heater 22 are suspended by the energizing members 16 and 17 and thereby fixed and electrically connected to the ceramic circuit board 15. The element unit 20 includes a temperature sensor for measuring the temperature of the first heater 22, and the temperature sensor is formed into a prescribed pattern on the surface of the substrate 21 on which the first heater 22 is disposed.

The substrate 21 may be, for example, a ceramic substrate. The detection element 23 may be formed, for example, as a mixed potential NOx (nitrogen oxide) sensor including a solid electrolyte layer and a pair of electrodes disposed on surfaces of the solid electrolyte layer. The first heater 22 has a meandering pattern.

In the present embodiment, the ceramic circuit board 15, the lid 11, the lower case 12, the seal members 13 and 14, and the element unit 20 in which the detection element 23 and the first heater 22 are disposed on the substrate 21 are integrated and form the “first unit” of the invention, to thereby constitute the sensing section 10.

A plurality of conductive pads 15p are disposed on the front and back sides of the base end portion 15e of the ceramic circuit board 15 and are electrically connected to the detection element 23 and the first heater 22 through lead portions and the energizing members 16 and 17. Conductive pads 15p on the back side of the ceramic circuit board 15 are not illustrated. An electric signal outputted from the detection element 23 is outputted outside the breath sensor through conductive pads 15p formed on the back side of the ceramic circuit board 15, and electric power is externally supplied to the first heater 22 through conductive pads 15p formed on the front side of the ceramic circuit board 15 to energize the first heater 22 and thereby generate heat.

As shown in FIG. 2, a tubular separator 19b is disposed on the forward end side of the tubular cassette connector 19, and a plurality of spring terminals 19c are held in a plurality of through holes of the tubular separator 19b. Therefore, when the base end portion 15e of the ceramic circuit board 15 is inserted into the cassette connector 19, the spring terminals 19c come into elastical contact with the conductive pads 15p of the base end portion 15e and are thereby electrically connected to the conductive pads 15p. Bare forward ends of the lead wires 19a are crimped and fixed to ends of the spring terminals 19c. The rear ends of the lead wires 19a are connected to an unillustrated female connector, and the lead wires 19a are thereby connected to an external device.

Referring next to FIG. 5, the conversion section 30 will be described.

The conversion section 30 includes: a rectangular plate-shaped upper lid 31; a rectangular frame-shaped spacer 33a1; a rectangular plate-shaped upper catalyst support 35a1 having opposite surfaces coated with a catalyst 41; a spacer 33a2; a rectangular plate-shaped upper catalyst support 35b1 having a surface (the surface facing upward in FIG. 5) coated with a catalyst 42; a heater substrate 50 having a rectangular plate-shaped main body and a narrow width base end portion 50e protruding from one edge of the rectangular main body; a rectangular plate-shaped lower catalyst support 35b2 having a surface (the surface facing downward in FIG. 5) coated with the catalyst 42; a spacer 33a3; a rectangular plate-shaped lower catalyst support 35a2 having opposite surfaces coated with the catalyst 41; a spacer 33a4; and a rectangular plate-shaped lower lid 32. These components are stacked in the above order from top to bottom in FIG. 5.

The spacers 33a1 to 33a4 have the same shape and may be collectively referred to as spacers 33a. The upper catalyst support 35a1 and the lower catalyst support 35a2 have the same shape and may be collectively referred to as catalyst supports 35a. Similarly, the upper catalyst support 35b1 and the lower catalyst support 35b2 have the same shape and may be collectively referred to as catalyst supports 35b.

The above components 31, 32, 33a, 35a, 35b and 50 are formed of, for example, a ceramic and are hermetically bonded and stacked with, for example, a glass or inorganic adhesive layer therebetween.

Since the upper lid 31 and the lower lid 32 have the same shape, only the lower lid 32 will be described. The lower lid 32 includes a rectangular plate having a through hole 32h and a pipe 32b that is attached to the through hole 32h so as to extend therethrough. The pipe 32b protrudes from the through hole 32h to the outside and is bent 90° along the plate surface of the lower lid 32, and the bent end extends beyond the peripheral edge of the lower lid 32 toward the base end portion 50e of the heater substrate 50. The upper lid 31 is similarly configured.

In the present example, a pipe 31a attached to the upper lid 31 serves as an introduction pipe for the breath G, and the pipe 32b serves as a discharge pipe.

The catalyst 41 is applied to opposite surfaces of the upper catalyst support 35a1. Specifically, the catalyst 41 is applied to approximately rectangular regions corresponding to the internal spaces of the spacers 33a1 and 33a2, and the upper catalyst support 35a1 has a slit-shaped opening 35s adjacent to edges of the catalyst 41 (the left edges in FIG. 5). The breath G introduced from the pipe 31a comes into contact with the catalyst 41 on the upper side within the internal space of the spacer 33a1, passes through the opening 35s, and then comes into contact with the catalyst 41 on the lower side within the internal space of the spacer 33a2.

The catalyst 42 is applied to one surface of the upper catalyst support 35b1 (the surface facing upward in FIG. 5). Specifically, the catalyst 42 is applied to an approximately rectangular region corresponding to the internal space of the spacer 33a2, and the upper catalyst support 35b1 has a circular hole-shaped opening 35h at the center of one edge of the catalyst 42 (the upper right edge in FIG. 5). The breath G comes into contact with the catalyst 42 within the internal space of the spacer 33a2 and then flows downward through the opening 35h.

The other surface of the upper catalyst support 35b1 is in contact with the heater substrate 50. When a second heater 51 having a meandering pattern is formed on the front surface of the heater substrate 50 and generates heat, the upper catalyst support 35b1 and the catalyst 42 are heated to a second temperature different from the first temperature through the heater substrate 50. A temperature sensor (not shown) for detecting the heating temperature of the second heater 51 is formed into a prescribed pattern on the back surface of the heater substrate 50. A circular hole-shaped opening 50h aligned with the opening 35h is formed in the heater substrate 50, and the breath G passing through the opening 35h flows downward through the opening 50h.

When the catalyst 42 is heated, the breath G in the internal space of the spacer 33a2 to which the catalyst 42 is exposed is heated, and the catalyst 41 exposed to the internal space of the spacer 33a2 is also heated. The heat of this catalyst 41 is also transferred to the catalyst 41 on the opposite surface (the upper surface) through the upper catalyst support 35al.

Useful catalysts 41 and 42 may be PtY catalysts that convert a gas component contained in the breath G to a specific component, e.g., convert NO in the breath G to NO₂.

In the present embodiment, the upper lid 31, the lower lid 32, the upper catalyst supports 35a1 and 35b1 (including the catalysts 41 and 42), the lower catalyst supports 35a2 and 35b2 (including the catalysts 41 and 42), the heater substrate 50 on which the second heater 51 is disposed, and the spacers 33a1, 33a2, 33a3 and 33a4 are integrated and form the “second unit” of the invention, to thereby constitute the conversion section 30.

A plurality of conductive pads 50p are disposed on the front and back surfaces of the base end portion 50e of the heater substrate 50 and are electrically connected to the second heater 51 and the temperature sensor (not shown) through lead portions. The second heater 51 is energized by electric power externally supplied through the conductive pads 50p to thereby generate heat.

As shown in FIG. 2, a tubular separator 39b is disposed on the forward end side of the tubular cassette connector 39, and spring terminals 39c are held in a plurality of insertion holes of the tubular separator 39b. Therefore, when the base end portion 50e of the heater substrate 50 is inserted into the cassette connector 39, the spring terminals 39c come into elastic contact with the conductive pads 50p of the base end portion 50e and are thereby electrically connected to the conductive pads 50p. Bare forward ends of the lead wires 39a are crimped and fixed to ends of the spring terminals 39c. The rear ends of the lead wires 39a are connected to an unillustrated female connector, and the lead wires 39a are connected to an external device.

Returning to FIG. 5, the lower catalyst support 35b2 is in contact with the lower surface of the heater substrate 50 (the surface facing downward in FIG. 5), and the catalyst 42 is applied to an approximately rectangular region on the lower surface of the lower catalyst support 35b2 (the surface facing downward in FIG. 5), as in the case of the upper catalyst support 35b1.

The lower catalyst support 35b2, the spacer 33a3, the lower catalyst support 35a2, the spacer 33a4, and the lower lid 32 that are on the lower side of the heater substrate 50 and the upper catalyst support 35b1, the spacer 33a2, the upper catalyst support 35a1, the spacer 33a1, and the upper lid 31 that are on the upper side of the heater substrate 50 are symmetric with respect to the plate surface of the heater substrate 50. The components on the lower side have substantially the same functions as the components on the upper side, and their detailed description will be omitted.

The breath G flowing downward through the opening 50h and an opening 35h of the lower catalyst support 35b2 comes into contact with the catalyst 42 within the internal space of the spacer 33a3 and then comes into contact with the catalyst 41 on the upper side of the lower catalyst support 35a2. Then the breath G passes through an opening 35s, comes into contact with the catalyst 41 on the lower side of the lower catalyst support 35a2 within the internal space of the spacer 33a4, and is discharged from the pipe 32b.

In the manner described above, the breath G is brought into contact with the catalysts heated to the second temperature, and the gas component (specifically, NO) contained in the breath G is converted to the specific component (specifically NO₂).

Returning to FIGS. 2 and 3, the conversion section 30 is oriented with its stacking direction aligned with a horizontal direction (the left-right direction in FIG. 3), is covered with an upper heat insulating member 71 from above and with a lower heat insulating member 72 from below, and contained in the internal space 93r of the base 93. The sensing section 10 is oriented with its stacking direction aligned with a vertical direction (the top-bottom direction in FIG. 3) and is contained in the internal space 92r of the upper case 92 with a sheet-shaped heat insulating member 73 disposed below the sensing section 10.

The upper heat insulating member 71, the lower heat insulating member 72, and the sheet-shaped heat insulating member 73 are formed, for example, of glass fibers.

The upper heat insulating member 71, the lower heat insulating member 72, and the sheet-shaped heat insulating member 73 correspond to the “heat insulating member” of the invention.

Sub-pipes 85, 84 and 83 are connected to the introduction pipe 31a of the conversion section 30, and one end of the main pipe 60 is connected to the discharge pipe 32b through a sub-pipe 86. The other end of the main pipe 60 is connected to the introduction pipe 12a of the sensing section 10 through a sub-pipe 82, and the sub-pipe 81 is connected to the discharge pipe 12b.

As described above, the first unit constituting the sensing section 10 and the second unit constituting the conversion section 30 are in communication with each other through the main pipe 60 that allows the breath G to pass therethrough. The breath G enters the conversion section 30 through the sub-pipe 85, then enters the sensing section 10 through the main pipe 60, and is discharged to the outside through the sub-pipe 81.

As described above, the main pipe 60 includes at least one bent portion (four bent portions in the present example, i.e., the bent portions 60a to 60d).

As described above, heat is applied to the conversion section 30 through the second heater 51 in order to activate the catalysts. However, since the first unit constituting the sensing section 10 and the second unit constituting the conversion section 30 are formed as single units and are in contact with each other through the upper heat insulating member 71, the lower heat insulating member 72 and the sheet-shaped heat insulating member 73, the influence of the heat on the sensing section 10 is reduced. Consequently, the accuracy of detection of the specific component by the sensing section 10 can be improved.

The main pipe 60 is bent at the bent portions 60a to 60d. Therefore, the path length of the main pipe 60 that is disposed outside the second unit is longer than that of an unbent pipe. In this case, the breath G heated in the conversion section 30 is easily cooled in the main pipe 60, and this can also reduce the influence of the heat applied to the conversion section 30 on the sensing section 10.

The present invention is not limited to the above-described embodiment and encompasses various modifications and equivalents within the spirit and scope of the invention.

For example, in the above embodiment, the first unit constituting the sensing section 10 and the second unit constituting the conversion section 30 are in contact with each other through the heat insulating members (the upper heat insulating member 71, the lower heat insulating member 72, and the sheet-shaped heat insulating member 73). However, the first unit (the sensing section 10) and the second unit (the conversion section 30) may be disposed inside the main body 90 so as to be separated from each other with a gap therebetween.

The main pipe 60 may be disposed inside the main body 90 without being exposed outside the main body 90. In this case, the breath sensor is less susceptible to external perturbations (such as wind) than a breath sensor including a pipe exposed outside of the casing. Therefore, the temperature of the cooled gas is stabilized, so that the output can be stabilized.

The shapes, materials, etc., of the breath sensor, i.e., the main body, sensing section, conversion section, and main pipe included in the breath sensor, are not limited to those in the above embodiment. No limitation is imposed on the type, etc., of the sensing section. No limitation is imposed on the placement positions of the sensing section and the conversion section within the main body, and no limitation is imposed on the placement position of the main pipe.

In the above embodiment, the first unit constituting the sensing section 10 is formed through use of the lower case 12, the lid 11, etc. However, like the second unit constituting the conversion section 30, the first unit may be formed by a ceramic stacked structure. In the above embodiment, the second unit constituting the conversion section 30 is formed by a ceramic stacked structure. However, like the first unit constituting the sensing section 10, the second unit constituting the conversion section 30 may be formed of a metal-made case containing catalysts. No particular limitation is imposed on the forms of the first and second units, so long as the sensing section 10 and the conversion section 30 are provided as separate units.

In the example shown in the above embodiment, the main pipe 60 includes the four bent portions 60a to 60d. However, the main pipe 60 may have any structure, so long as it has a bent portion. The main pipe 60 may have a structure in which the main pipe 60 is bent a plurality of times in the longitudinal direction in a meandering manner or a structure in which the main pipe 60 is bent a plurality of times into a helical shape.

The invention has been described in detail with reference to the above embodiments. However, the invention should not be construed as being limited thereto. It should further be apparent to those skilled in the art that various changes in form and detail of the invention as shown and described above may be made. It is intended that such changes be included within the spirit and scope of the claims appended hereto.

What is claimed is:

1. A breath sensor comprising:
a main body into which breath can be introduced;
a conversion section that converts a gas component contained in the breath to a specific component; and
a sensing section that detects the specific component contained in the breath passing through the conversion section, the conversion section and the sensing section being disposed inside the main body,
wherein the sensing section constitutes a first unit including an integrated detection element and a first heater, the detection element having electric characteristics that vary depending on the concentration of the specific component, and the first heater heating the detection element to a first temperature,
wherein the conversion section constitutes a second unit including an integrated catalyst and a second heater, the catalyst converting the gas component contained in the breath, the second heater heating the catalyst to a second temperature different from the first temperature,
wherein the first unit and the second unit are disposed inside the main body so as to be separated from each other with a gap therebetween, or are in contact with each other via a heat insulating member,
wherein the first unit and second unit are in communication with each other through a pipe that allows the breath to pass therethrough, and
wherein the pipe is partially bent.

2. The breath sensor as claimed in claim 1, wherein the pipe is disposed inside the main body without being exposed outside the main body.
